(19) United States
(12) Patent Application Publication
(10) Pub. No.: US 2025/0235616 A1
(43) Pub. Date: Jul. 24, 2025

(54) Supplemental device for a medicament delivery device

(71) Applicant: SHL Medical AG, Zug (CH)

(72) Inventor: Daniel Säll, Nacka Strand (SE)

(21) Appl. No.: 18/697,890

(22) PCT Filed: Sep. 28, 2022
(86) PCT No.: PCT/EP2022/076937

§ 371 Date: Apr. 2, 2024

(30) Foreign Application Priority Data
Oct. 8, 2021 (EP) 21201605.9

(51) Int. Cl. A61M 5/20; A61M 5/24; A61M 5/32; A61M 5/50

(52) U.S. Cl. CPC A61M 5/20; A61M 5/24; A61M 5/3202; A61M 5/5086; A61M 2205/3561; A61M 2205/8212

(57) ABSTRACT

The present disclosure provides a supplemental device attached to a removable cap of a medicament delivery device. The supplemental device includes a housing attachable to the removable cap and configured to accommodate a battery module. The battery module includes a battery and a switch operatively connected to the battery module. The supplemental device further includes an activation clip operatively connected to the battery module. The supplemental device further includes a communication module connectable to the battery module that is configured to transmit data to an external device. The supplemental device further includes an insulation strip including a first portion that is slidably arranged between the switch and the activation clip and a second portion that is attachable to a housing of the medicament delivery device. The supplemental device is initially in a first state where the communication module is prevented to receive power from the battery module and where a first or second movement of the removable cap relative to the housing changes the supplemental device to a second state where the battery provides power to the communication module.

### CROSS-REFERENCE TO RELATED APPLICATIONS

The present application is a U.S. National Phase Application pursuant to 35 U.S.C. § 371 of International Application No. PCT/EP2022/076937 filed Sep. 28, 2022, which claims priority to European Patent Application No. 21201605.9 filed Oct. 8, 2021. The entire disclosure contents of these applications are herewith incorporated by reference into the present application.

### TECHNICAL FIELD

The present invention relates to a supplemental device that attaches to a medicament delivery device, where the supplemental device can function as an information provider that is removable capable of transmitting unique information concerning the medicament delivery device. The supplemental device is activated by the removal of a covering from the medicament delivery device. The supplemental device can monitor the use of the medicament delivery device and can communicate with external smart devices.

### BACKGROUND

Medicament delivery devices, especially those designed for medicament delivery by self-administration, have been on the market for a number of years. In order for the devices to be handled by non-professionals, they have to be easy to use and intuitive. Further, since many of the medicaments are vital or at least very important to the patient there is a desire from physicians and other professionals to obtain information that the patients medicate according to prescribed schemes. The desired information could include the type of medicament, delivery times, dates, dose size, safety information, such as sterility. Also, of importance is whether the medicament or the device itself is counterfeit or has been tampered with. Likewise, information that could be beneficial to the physician concerns whether the medicament has been taken using the correct procedure and following instructions for use; that the medicament has been maintained at the prescribed temperature before and during medicament delivery; that the right injection depth has been used and that the correct injection speed has been used, when the medicament delivery device is an injector.

Systems for obtaining information from a medicament delivery device are known. For example, WO 2004/084116 discloses a system for presenting and distributing medication information, where a medicament delivery device is arranged with communication mechanisms which will enable communication with an external device such as a cellular or a mobile phone or a personal digital assistant (PDA). A preferred communication standard is Bluetooth. The medicament delivery device is arranged with a number of sensors for monitoring and registering e.g. a dose delivery sequence. The idea is then to use the functionality of the external device, such as its display, its processor, its keyboard, etc. instead of providing the medicament delivery device with such features. The transfer of the functionality to the external device will reduce the cost of the medicament delivery device in comparison with medicament delivery devices provided with such functionality.

However, a drawback with the solution according to WO 2004/084116 is that a Bluetooth circuit, or the like wireless communication systems such as ANT or ZigBee, is built into the housing of the medicament delivery device. The communication system with its battery to power the circuit requires a dedicated space in a medicament delivery device. Manipulation of existing device designs to accommodate an add-on battery module may trigger unforeseen regulatory problems. Another issue with known data collection devices is that the power source is directly connected to the electronic circuit at the time the battery module is manufactured and attached to the medicament delivery device. This can cause premature battery drain. Additionally, such systems require the user to perform a separate and specific step of activating the battery module, which may not always be performed successfully.

It is thus not so easy to modify existing designs or to easily provide existing designs with added functionality that communication systems can provide. As such, there is a need to provide an activation system that is automatic and is performed prior to a breach of the sterility barrier, as part of the normal use of the medicament delivery device, providing an detection mechanism activated by the removal sequence of the protective unit before the sterility barrier is removed, thereby activating the supplemental device for recording and signalling the breach of the sterility barrier of the medical delivery device.

### BRIEF DESCRIPTION

The present disclosure is applicable to a number of medicament delivery devices may include a number of devices capable of delivering certain doses of medicament to a user, such as e.g. injection devices with or without injection needles, inhalers of all kinds, such as powder, aerosol driven, gas, nebulizers having mouth or nasal pieces, dispensers for medicament in tablet form. The medicament delivery devices may be of either disposable type or re-usable type and may be provided with medicament containers suitably arranged for specific medicaments in specific forms. Further the medical devices, include, but are not limited to, devices that automatically, semi-automatically or manually deliver one or more doses of medicament.

In the following description, the wording smart device will be used. In this context, smart devices may include electronic devices that are provided with processors or processing circuitry that are capable of running computer programs as well as storage space to store programs as well as data retrieved from different external sources. The processing circuitry may for example use any combination of one or more of a suitable central processing unit (CPU), multiprocessor, microcontroller, digital signal processor (DSP), application specific integrated circuit (ASIC), field programmable gate arrays (FPGA) etc., capable of executing any herein disclosed operations concerning detection and or transmission of data. It is further to be understood that the smart devices are provided with communication systems that are capable of communicating with data networks in order to access different databases. It is to be understood that databases may be accessed via the internet, so called cloud services, and/or databases that are connected directly to and accessed via local area networks. It is further to be understood that the smart devices in this context comprise some sort of human-machine interface for two-way communication. The human-machine interface may comprise displays, keyboards, microphones, loudspeakers, I/O-ports for connection of peripherals. Further the smart devices may be provided with antennas for wireless communication with the networks. Also, the smart devices may be arranged with receiving and transmitting mechanisms capable of communicating with NFC tags as well as programs capable of establishing and handling the communication with the NFC tags. Also, the smart devices may be arranged with receiving and transmitting mechanisms capable of communicating via Li-Fi (light-fidelity) technology.

The aim of the present invention is to provide an activation mechanism for a supplemental device that is attachable to an existing medicament delivery device, where the attachment does not require that the existing design of the medicament delivery device be modified or otherwise changed. A particularly preferred supplemental device is an information provider device that can be used on and with a number of different medicament delivery devices, and in particular configured for use on medicament delivery devices for self-administration of medicaments. Preferably the medicament delivery devices arranged with such information providers could be used with conventional external smart devices common on the market and used by the majority of patients that handle medicament delivery devices for self-administration.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the medicament delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

As used herein, the term “medicament” is meant to encompass any medicament-containing flowable medicine capable of being passed through a delivery means such as a cannula or hollow needle in a controlled manner, such as a liquid, solution, gel or fine suspension. Representative medicaments include pharmaceuticals such as peptides, proteins, and hormones, biologically derived or active agents, hormonal and gene-based agents, nutritional formulas and other substances in both solid (dispensed) or liquid form. In the description of the exemplary embodiments reference will be made to the use of insulin. Correspondingly, the term “subcutaneous” infusion or injection is meant to encompass any method of transcutaneous delivery to a subject.

In the present disclosure the term “module” is meant to encompass a self-contained unit or item, such as an electronic component or an assembly of electronic components and associated wiring, which itself performs a defined task and can be linked with other such unit to form a larger system.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

The term automatic in conjunction with medical injection device means that, the injector device is able to perform the injection without the user of the device delivering the force needed to expel the medicament during delivery. The force is delivered automatically by a resilient force like a spring or an electric motor or any other suitable means for providing the delivery force.

A first aspect of the invention concerns a supplemental device attached to a removable cap of a medicament delivery device. The supplemental device comprises a housing attachable to the removable cap, wherein the housing is configured to accommodate a battery module, an activation clip connected to the battery module, a switch having a first surface connected to the battery module and a second surface which is connectable to the activation clip and when connected creates a contact area, and a communication module connectable to the battery module or included as part of the battery module, and configured to transmit data to an external device, where the data comprises information directly related to a status of the switch. The supplemental device further comprises an insulation strip, having a first portion which has a predetermined area that is larger than the contact area between the switch and the activation clip, and a second portion which is attachable to a housing of the medicament delivery device which extends along a longitudinal axis between a proximal and distal end;

wherein the supplemental device is initially in a first state S1 when the first portion of the insulation strip is slidably arranged between switch and the activation clip such that the communication module is prevented to receive power from the battery module and where the removable cap is removed by a first and/or second movement M1, M2 towards the proximal end relative to the housing of the medicament delivery device. The removal of the removable cap changes the supplemental device to a second state S2 when the first portion of the insulation strip is removed from the contact area between the switch and the activation clip thereafter the battery module provides power to the communication module.

An advantage of the supplemental device is that any pre-existing medicament delivery device can be combined and upgraded with such a smart device. Further, as the power supply and power consumption of the supplemental device starts only when a user uncaps the device, the battery size can be small and limited to the expected time of use of the device and not arranged for a long shelf life.

Optionally the insulating strip is flexible or adaptable to a shape of an outer and/or inner surface of the medicament delivery device, or the insulating strip comprises a shape that matches the outer and/or inner surface of the medicament delivery device.

Optionally, the insulating strip extends along the longitudinal direction, wherein the first portion is arranged within the housing of the supplemental device and the second portion extends towards the distal end of the housing of the supplemental device.

Optionally, the first movement is a linear movement of the removable cap in relation to the housing of the medicament delivery device and towards the proximal end.

An advantage of the insulating strip being partially in the supplemental device and partially attached to the housing, is that the supplemental device is only activated when the removable cap is completely taken away from the device, i.e. when the removable cap performs a linear movement M1 in proximal direction. Thus, a slightly longitudinal movement of the removable cap does not activate the supplemental device.

Alternatively, the insulating strip extends in a circumferential direction around the housing of the medicament delivery device, wherein the first portion is arranged within the housing of the supplemental device and the second portion extends in circumferential direction out of the housing of the supplemental device.

Optionally, the second movement M2 is first a rotational movement and then a linear movement or vice-versa or a helical movement or a linear movement of the removable cap in relation to the housing of the medicament delivery device and towards the proximal end.

An advantage of the circumferentially arranged insulating strip is that medicament delivery devices which uncap with a twist and pull movement can easily be upgraded with the supplemental device.

An advantage is that an accidental breach of the sterility barrier is detected by the supplemental device. For example, patients with mental disorders who may forget to administer a dose of the medicament after uncapping the medical delivery device, can be reminded by an external system which has received the information from the communication module. Also sterility breach of an individual medicament delivery device within a large stock of medicament delivery devices can be monitored in this way.

Optionally, the supplemental device further comprises an adhesive pad configured to securely attach and hold supplemental device to the cap.

An advantage is high adaptability to a large number of medicament delivery devices and the facility to add smart device to existing models.

Optionally, the battery module or the communication module comprises one or more sensors configured to detect a motion or a status of at least one of the components of the medicament delivery device.

An advantage is the monitoring of the medicament delivery and or the mixing process of the medicament components of a medicament delivery device.

Optionally, the supplemental device further comprises a sensor module connectable to any other module and wherein said sensor module comprises one or more sensors configured to detect a motion or status of at least one of the components of the medicament delivery device.

An advantage of the modularity is that the sensor component can be reused easily and rearranged in a other supplemental device. For example when the battery module has come to an end of life and has to be disposed, the sensor module can be separated from the battery module from the supplemental device and added to a new battery module.

Optionally, any of the modules is removable from the housing and is reusable.

Optionally the battery module or the communication module comprises a recorder configured to obtain and store data information about the medicament delivery device.

An advantage is that specific data about the medicament, the medication details of the patient or the environmental data can be collected and transmitted. For example, the medicament intake of a patient can be monitored, or if a medicament has to be stored or used at a specific temperature, and this was not applied to the medicament delivery device, the user can be notified. Or specific information about the batch or information about the specific therapeutic administration plan of the patient can be stored thereon.

Optionally, the data transmission by the communication module starts when the recorder receives power from the battery.

Optionally, the supplemental device further comprises a logging module connectable to any other module or included as part of any other module, and wherein the logging module is configured to start tracking motion of the medicament delivery device when the removable cap is removed.

Optionally, the supplemental device further comprises a memory module connectable to any other module or included as part of any other module, and wherein the memory module is configured to store the data and the communication module is configured to transmit the data wirelessly to the external device.

A second aspect concerns medicament delivery device comprising a housing, a medicament container positioned within the housing; and a dose delivery outlet accessible through a terminal end of the housing, a removable cap attached to the housing such that the dose delivery outlet is prevented from being accessible unless the removable cap is fully removed from the medicament delivery device; and a supplemental device according to any one of the preceding options.

These and other aspects of and advantages with the present invention will become apparent from the following detailed description and from the accompanying drawings.

### BRIEF DESCRIPTION OF THE DRAWINGS

Embodiments of the invention will now be described by way of example only and with reference to the accompanying drawings, in which:

FIG. 1A shows a medicament delivery device with the supplemental device attached to it.

FIG. 1B shows a side view of the supplemental device of FIG. 1A.

FIG. 1C shows the supplemental device of FIG. 1A in a three-dimensional perspective view.

FIG. 2A to 2C show the medicament delivery device with the attached supplemental device of FIG. 1A in a top view, a lateral view and a back view.

FIGS. 3A to 3D show the supplemental device in a top view, lateral view, perspective back view and back view.

FIGS. 4A to 4C show an alternative the supplemental device with further segmentations of the parts. FIGS. 4D and 4E show a distal top and proximal top view of the supplemental device.

FIG. 5 shows an exploded view of the supplemental device.

FIG. 6A shows the supplemental module attached to the medicament delivery device with the communication module taken away.

FIG. 6B shows the arrangement of the activation clip, the insulation strip, the switch and the adhesive pad of the supplemental device with respect to the medicament delivery device in the first state S1.

FIG. 6C shows the arrangement of the activation clip, the insulation strip, the switch and the battery module in the first state S1.

FIG. 7A shows the supplemental device attached to a medicament delivery device with an insulating strip arranged in circumferential direction in an first state S1.

FIG. 7B shows the device of 7A with a twist and pull movement of the removable cap of the medicament delivery device in the second state (S2).

### DETAILED DESCRIPTION

Various modifications to the embodiments described are possible and will occur to those skilled in the art without departing from the invention which is defined by the following claims.

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

Further, in the following description, the wording medicament delivery device will be used. In this context, medicament delivery devices may include a number of devices capable of delivering certain doses of medicament to a user. The medicament delivery devices may be of either disposable type or re-usable type and may be provided with medicament containers suitably arranged for specific drugs in specific forms.

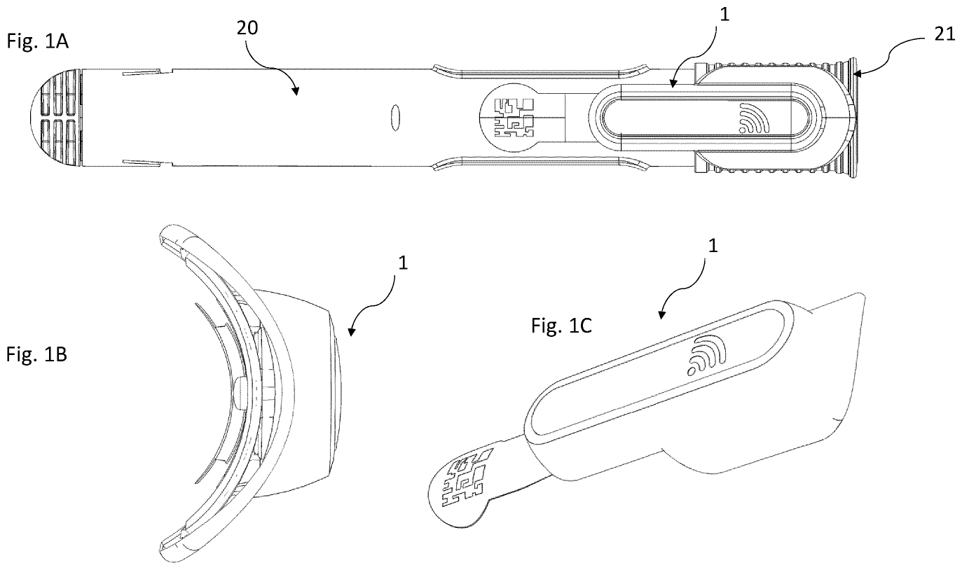

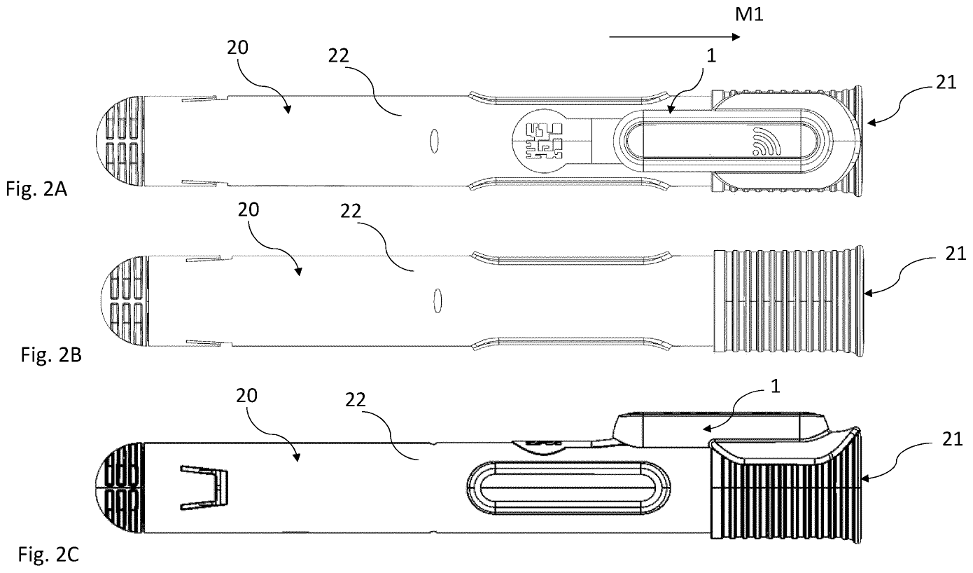

FIG. 1A and FIGS. 2A-2C show the supplemental device 1 as attached to a medicament delivery device 20. In this example the medicament delivery device 20 has a tubular elongated body extending from a distal to a proximal end. The medicament delivery device comprises a housing 22 arranged to accommodate a medicament container, an activation mechanism, a medicament delivery member. At the proximal end, protecting the medicament delivery member is a removable cap 21. The supplemental device 1, comprises a housing 11 extending longitudinally from the removable cap towards the distal end of the medicament delivery device 20. In the example shown in FIGS. 1A and 2A-2C the housing 11 of the supplemental device partially extends above the longitudinal dimension of the removable cap 21, such that a portion of the housing 11 is arranged on the housing 22 of the medicament delivery device 20.

In an alternative example not shown in the figures the housing of the supplemental device does not extend beyond the removable cap 21. For example, the housing of the supplemental device may have a c-clip shape which embraces the removable cap circumferentially. Alternatively, the housing adopts the cross-sectional form of the cap, i.e. it can be added on the proximal end or top of the cap.

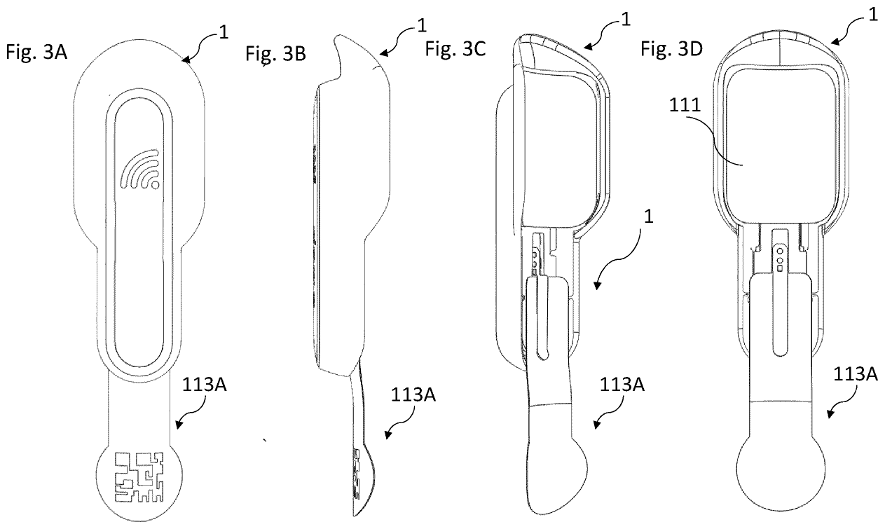

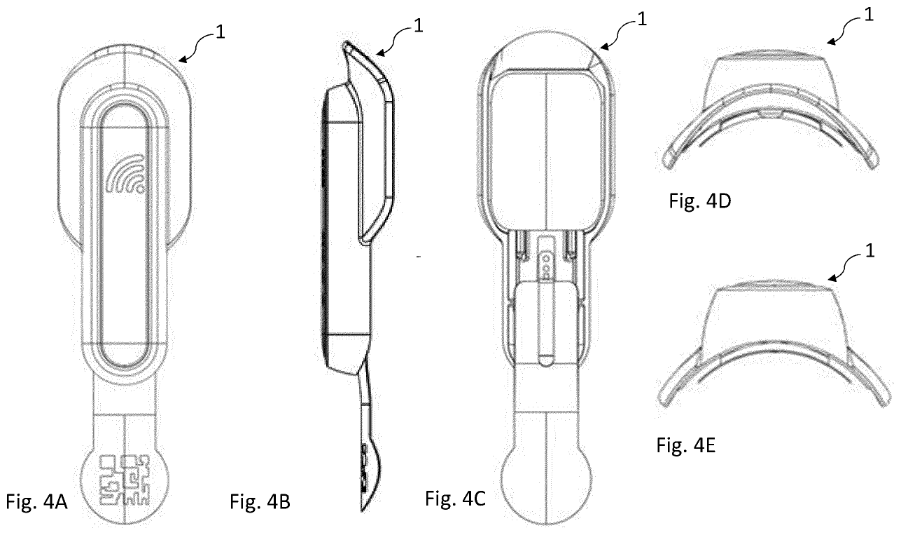

Attaching the housing 11 of the supplemental device 1 to the removable cap 21 of the medicament delivery device 20 comprises in the present example an adhesive pad 111 as shown in FIGS. 3D, 5 and 6B. The supplemental device is adhesively attached to the removable cap 21. The adhesive pad 111 preferentially comprises an adhesive glue configured to glue reversibly or irreversibly to the cap. Alternatively the adhesive pad 111 comprises a suction mechanism, for example a surface with micro suction cups, for attaching reversibly the supplemental device 1 to the removable cap 21. Even alternatively the adhesive pad 111 is a magnetic pad, which magnetically attaches the supplemental device to the cap.

In the above alternative example with a c-clip shaped housing of the supplemental device, the attachment pad may have the form of a resilient c-clip which holds the supplemental device with the resilient force alone, or in combination with any adhesive example of above, in place attached to the cap.

In the above example with a cross-sectional cap shape of the supplemental device, the various versions of the adhesive pad i.e. glue, suction or magnetic type can be applicable, further the supplemental device may further comprise threaded means for attaching the supplemental device to the cap. In this case the adhesive pad or the threaded means alone or in combination may be used.

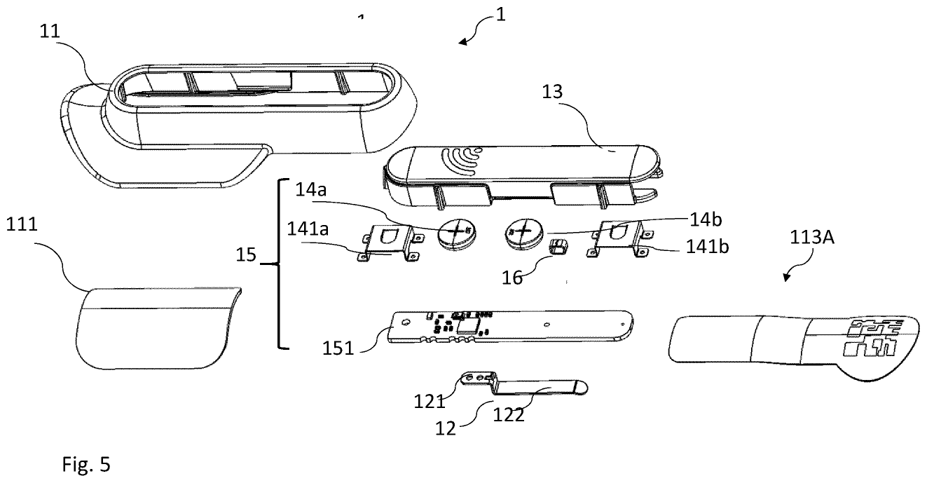

The housing 11 of the supplemental device is configured to receive various modules. As is illustrated in FIG. 5, the housing 11 receives a communication module 13, a battery module 15 comprising a circuit board 151 arranged to the circuit board one or more batteries 14a, 14b, optionally battery mounting structure 141a, 141b, a switch 16 and an activation clip 12. Optionally the supplemental device comprises sensor modules, not shown in the figures, for example the sensor modules comprising sensors for temperature, humidity, movement, light, sound or vibrational, or electric or magnetic field sensors.

Optionally the supplemental device comprises data storage means for storing data relative to the medicament delivery device, the patient the medicament or any other relevant useful data. For example, it may further store a computer program with instructions for the communication module 13.

For example, when the communication module 13 is designed for a large variety of medicament delivery devices and medicaments, respective computer programs related to the specific medicament delivery device or the medicament or the patient may be stored in storage means dedicated to the medicament delivery device type or therein contained medicament. To give an example, some type of medicaments have to be stored at a certain temperature but have to be administered at a temperature different from the storage temperature. In this case a temperature sensor transmits the temperature data to the data storage device, and the data storage device transmits the data and computer program to the communication module which upon execution of the computer program sends a notification to the user, that the storage temperature has not been breached previously and that the temperature for administration has been reached. The notification sent to the user is sent for example via the wireless communication to a smart phone, a computer or a separate displaying device, or any other appropriate displaying device.

Optionally the communication module 13 comprises further a display module showing notifications to the user. The display module not shown in the figures, can alternatively be attached to or integrated in the medicament delivery device. The display module in this example is then connectable to the communication module 13 of the supplemental device. Optionally the display module further comprises additional signalling elements for tactile, visual, and/or audible signalling. For example, a vibration generator for the tactile signalling and speakers for audible signalling.

In some examples the battery module 15 and the communication module 13 are designed as a single module which for example can be reused together and reassembled with new batteries, switch and activation clip.

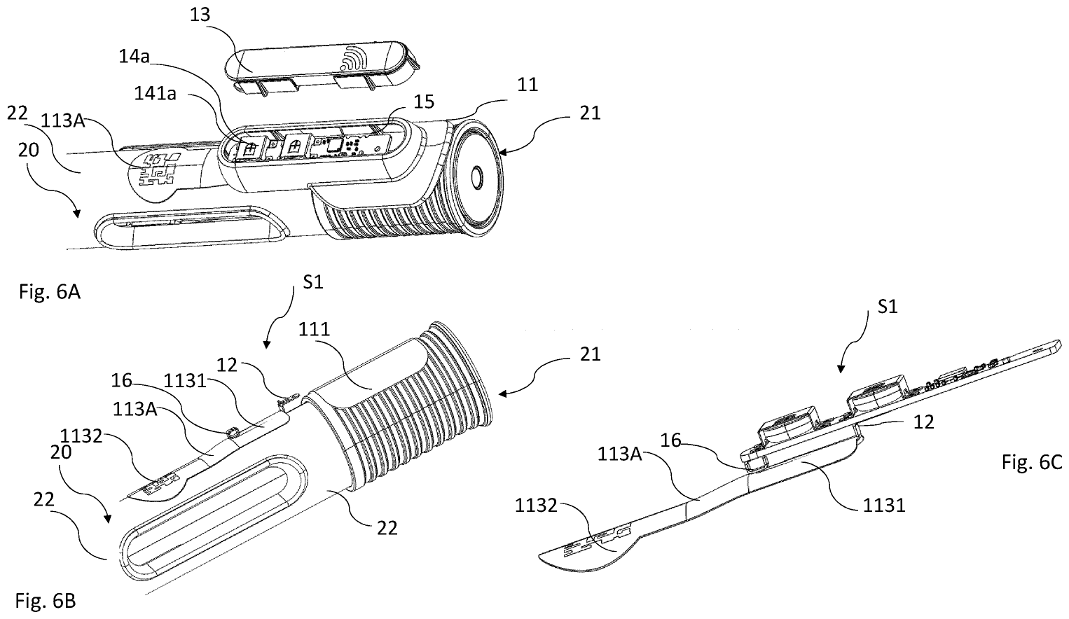

As is illustrated in FIGS. 3A to 3D the supplemental device is arranged with an insulating strip 113A, a first portion of the insulating strip is arranged within the housing 11 of the supplemental device as shown in FIGS. 3C and 3D and a second portion of the insulating strip is configured to be attached to the housing of the medicament delivery device 20, this can be seen in FIGS. 2A and 6A for example. The activation clip 12 is shaped such that a first portion 121 of the clip is operatively connected to the battery module 15, further a second portion 122 is formed such that it provides a supporting part for the insulating strip 113A, 113B in this example the second portion 122 extends longitudinally from the connection to the battery module 15 towards the spaced apart switch 16, the switch 16 also is connected to the battery module 15 shown in FIG. 6C. The first portion 1131 of the insulating strip 113A, 113B is arranged between the second portion 122 of the activation clip 12 and the switch 16 illustrated in FIGS. 6B and 6C. In the first state (S1) of the supplemental device 1 the insulating strip 113A, 113B prevents the communication module to receive power from the battery module. In order to activate the communication module 13, the insulating strip 113A, 113B needs to be moved such that a contact between the switch and the activation clip is established. This is achieved when a first or second movement (M1, M2) of the removable cap 21 relative to the housing 22 of the medicament delivery device changes the supplemental device 1 to a second state S2 when the first portion 1131 of the insulation strip is removed from the contact area between the switch 16 and the activation clip 12 such that the battery 14a, 14b provides power to the communication module 13. When the communication module 13 receives power from the battery, the communication module can send notifications to an external device or a display for example. Further, the battery module may directly or via the communication module provide power to the sensor module. In the example as shown in the FIGS. 6A-6C the connection of the activation clip 12 to the battery module 15 is longitudinally spaced apart from the switch 16, this provides a longitudinal travel distance for the insulating strip 113A, 113B before activation. Or in other words the removable cap 21 has to move (M1) a certain distance in a proximal direction, the travel distance is set by the initial position of the insulating strip in the second portion 122 of the activation clip 12. The maximal travel distance is thus the distance between position of the switch 16 and the position of the connection of the activation clip 12 to the battery module 15. An advantage is that the removable cap 21 accidentally can travel a certain distance in the proximal direction without that the insulating strip 113A, 113B moves beyond the switch 16 in distal direction. As cited above in the first state S1 of the supplemental device 1 the insulating strip 113A, 113B prevents the communication module to receive power from the battery module. The removal of the removable cap 21 moves the attached supplemental device 1 in a proximal direction, whereby the movement (M1,M2) can be any one of a linear, circumferential or helical movement or any combination of them. The displacement of the supplemental device 1 in proximal direction moves the switch 16 and the activation clip 12 in proximal direction thereby the insulating strip 113A, 113B moves out of the contact area of the switch and the activation clip, in other words the insulation strip 113A, 113B moved beyond the position of the switch 16 and allows the contact between the switch 16 and the activation clip 12. In this way an accidental activation is not possible. The second portion 1132 of the insulating strip 113A, 113B has an adhesive portion with which the insulating strip is attached to the housing 22 of the medicament delivery device 20, in other words when the supplemental device 1 is attached to the medicament delivery device 20, the housing 11 of the supplemental device 1 is attached to the removable cap 21 via the adhesive pad 111, and the insulating strip 113A, 113B is attached to the housing 22 of the medicament delivery device via the adhesive portion of the insulating strip 113A, 113B.

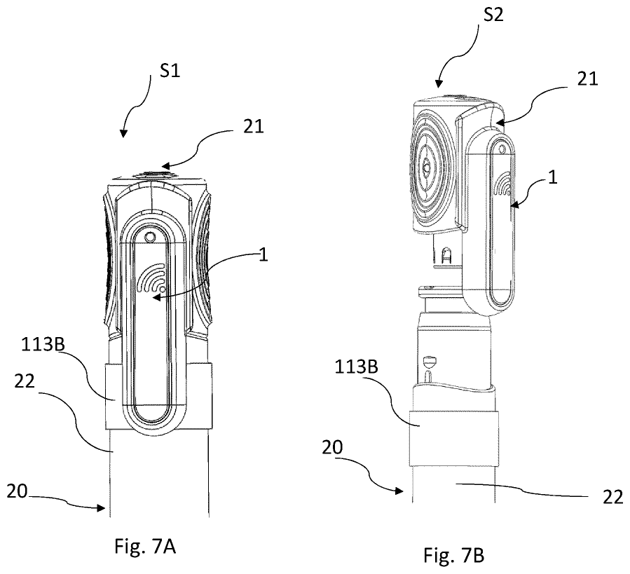

In an alternative example shown in FIGS. 7A and 7B the insulating strip is wrapped circumferentially around the housing 22 of the medicament delivery device 20 and the second portion 1132 of the insulating strip 113B is attached circumferentially at the housing 22 of the medicament delivery device. The removable cap 21 can be twisted before an activation occurs. In this way a movement M2 comprising first a twisting and then a pulling movement of the removable cap 21 is necessary to activate the supplemental device 1.

The second portion 1132 of the insulating strip further may comprise information on the external side which is visible to the user. A variety of tags can be envisaged, for example QR codes, RFID tags, temperature sensors (sensors changing colour if a threshold temperature was reached), holograms, labels etc.

In an alternative example not shown in the figures, the supplemental device is non releasably coupled to the cap, it can be integrated in the cap. In this example the supplemental device is arranged with an insulating strip. A first portion of the insulating strip is arranged within the housing 11 of the supplemental device i.e. in the cap, and a second portion of the insulating strip is configured to be attached to the housing of the medicament delivery device 20. In a further example the battery module comprising one or more batteries and optionally a battery mounting structure are integrated in the removable cap and the supplemental device is connected to the battery module in the removable cap electrically. In this way the supplemental device has a connection to an external battery module. The modular aspect of the herein described supplemental device has the advantage to combine the different modules for different needs, this allows an easy adaptation and modification of the device. Further, any pre-existing medicament delivery device can easily be upgraded with a smart device function without changing the underlying medicament delivery device. More functionalities and security can be added to pre-existing devices without modifying them. With the modular structure the supplemental device further can be reused entirely or in parts. With the activation triggered by the removable cap removal battery life and battery size can be optimized. This in turn lowers material costs of the device and allows for a more compact design of the supplemental device.

In a further example not shown in the figures the insulating strip is arranged such that after the removable cap 21 has been pulled away and the communication module is activated by the contact of the switch 16 with the activation clip 12, when the removable cap 2 is put back on the medicament delivery member the insulating strip is configured to be repositioned between the switch and the activation clip such that the power supply is interrupted again. In this way the activation mechanism is reusable, which might be an advantage for a multidose or reusable device.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope being indicated by the claims.

1-15. (canceled)

16. A supplemental device attached to a removable cap of a medicament delivery device, the supplemental device comprising:
a housing attachable to the removable cap, wherein the housing is configured to accommodate:
a battery module,
an activation clip connected to the battery module,
a switch having a first surface connected to the battery module and a second surface which is connectable to the activation clip and when connected creates a contact area; and
and a communication module connectable to the battery module or included as part of the battery module, and configured to transmit data to an external device, where the data comprises information directly related to a status of the switch,
the supplemental device further comprises an insulation strip, having a first portion which has a predetermined area that is larger than the contact area between the switch and the activation clip, and a second portion which is attachable to a housing of the medicament delivery device which extends along a longitudinal axis between a proximal and distal end,
wherein the supplemental device is initially in a first state when the first portion of the insulation strip is slidably arranged between switch and the activation clip such that the communication module is prevented to receive power from the battery module and where the removable cap is removed by a first and/or second movement towards the proximal end relative to the housing of the medicament delivery device, the removal of the removable cap changes the supplemental device to a second state when the first portion of the insulation strip is removed from the contact area between the switch and the activation clip thereafter the battery module provides power to the communication module.

17. The supplemental device of claim 16, wherein the insulating strip, is flexible or adaptable to a shape of an outer and/or inner surface of the medicament delivery device, or the insulating strip comprises a shape that matches the outer and/or inner surface of the medicament delivery device.

18. The supplemental device of claim 16, wherein the insulating strip extends along the longitudinal axis, wherein the first portion is arranged within the housing of the supplemental device and the second portion extends towards the distal end of the housing of the supplemental device.

19. The supplemental device of claim 18, wherein the first movement is a linear movement of the removable cap in relation to the housing of the medicament delivery device and towards the proximal end.

20. The supplemental device of claim 16, wherein the insulating strip extends in a circumferential direction around the housing of the medicament delivery device, wherein the first portion is arranged within the housing of the supplemental device and the second portion extends in circumferential direction out of the housing of the supplemental device.

21. The supplemental device of claim 20, wherein the second movement is first a rotational movement and then a linear movement or vice-versa or a helical movement or a linear movement of the removable cap in relation to the housing of the medicament delivery device and towards the proximal end.

22. The supplemental device of claim 16, wherein the supplemental device further comprises an adhesive pad configured to securely attach and hold the supplemental device to the removable cap.

23. The supplemental device of any one of the preceding claims, wherein the battery module or the communication module comprises one or more sensors configured to detect a motion or a status of at least one of the components of the medicament delivery device.

24. The supplemental device of claim 16, wherein the supplemental device, further comprises a sensor module connectable to any other module and wherein said sensor module comprises one or more sensors configured to detect a motion or status of at least one of the components of the medicament delivery device.

25. The supplemental device of claim 16, wherein any of the modules is removable from the housing and is reusable.

26. The supplemental device of claim 16, wherein the battery module or the communication module comprises a recorder configured to obtain and store data information about the medicament delivery device.

27. The supplemental device of claim 26, wherein the data transmission by the communication module starts when the recorder receives power from the battery.

28. The supplemental device of claim 16, wherein the supplemental device further comprises a logging module connectable to any other module or included as part of any other module, and wherein the logging module is configured to start tracking motion of the medicament delivery device when the removable cap is removed.

29. The supplemental device of claim 16, wherein the supplemental device further comprises a memory module connectable to any other module or included as part of any other module, and wherein the memory module is configured to store the data and the communication module is configured to transmit the data wirelessly to the external device.

30. A medicament delivery device comprising:
a housing;
a medicament container positioned within the housing;
a dose delivery outlet accessible through a terminal end of the housing;
a removable cap attached to the housing such that the dose delivery outlet is prevented from being accessible unless the removable cap is fully removed from the medicament delivery device; and
the supplemental device of claim 16.
